(19) United States
(12) Patent Application Publication
(10) Pub. No.: US 2009/0087487 A1
(43) Pub. Date: Apr. 2, 2009

(54) Paliperidone sustained release formulation

(76) Inventors: Michael Fox, Tel Aviv (IL); Simona Di Capua, Kfar Saba (IL); Ronny Reinberg, Modii'n (IL)

Correspondence Address: KENYON & KENYON LLP, ONE BROADWAY, NEW YORK, NY, 10004, US

(21) Appl. No.: 12/229,344

(22) Filed: Aug. 21, 2008

Related U.S. Application Data
(60) Provisional application No. 60/935,597, filed on Aug. 21, 2007.

(51) Int. Cl. A61K 9/22; A61K 31/519; A61P 25/00; A61K 9/14

(52) U.S. Cl. 424/468; 514/259.41; 424/501; 424/490

(57) ABSTRACT

The present invention provides sustained release dosage forms comprising Paliperidone and processes for preparing the same.

### CROSS REFERENCE TO RELATED APPLICATION

The present application claims the benefit of U.S. Provisional Application No. 60/935,597, filed Aug. 21, 2007, the disclosure of which is incorporated by reference.

The present invention relates to sustained release pharmaceutical compositions comprising Paliperidone or a salt thereof, and a process for preparing the same.

### BACKGROUND OF THE INVENTION

Paliperidone is described in U.S. Pat. No. 4,804,663. The paliperidone compound differs from risperidone and related prior art compounds described in U.S. Pat. Nos. 4,352,811 and 4,458,076 by its substitution on the 1-position of the piperidine moiety.

Paliperidone (CAS Registry No. 144598-75-4) has the chemical name 4H-Pyrido[1,2-a]pyrimidin-4-one, 3-[2-[4-(6-fluoro-1,2-benzisoxazol-3-yl)-1-piperidinyl]ethyl]-6,7,8,9-tetrahydro-9-hydroxy-2-methyl. Paliperidone is represented by the structural formula:

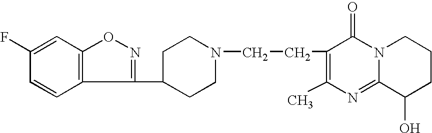

Paliperidone is practically insoluble in water, freely soluble in methylene chloride and soluble in methanol and 0.1 N hydrochloric acid. Additionally, since paliperidone has a long half-life of about one day, it is not a typical candidate for extended delivery. However, side effects such as anxiety, somnolence, dizziness, constipation, extrapyramidal symptoms, may be related to high blood plasma concentration levels restricting the ability to administer a single daily immediate release dose.

A published patent application, US 2004/0092534, discloses extended release formulations and methods for providing ascending rate of release of paliperidone utilizing a capsule-shaped tablet. The dosage form utilizes a semipermiable membrane surrounding a three layer core: the first layer contains low amounts of drug and an osmotic agent; the middle layer contains higher amounts of drug and without osmotic agent and the third layer is a push layer. In addition to the said structure of capsules shape tablet, there is at least one orifice which drilled through the membrane on the first drug layer end. All this capsule shaped tablet is designed to be a once-a day dosage form.

U.S. patent application publication No. US 2006/034927 relates also to a Paliperidone dosage form for sustained release of a drug comprising: a delay layer comprising (i) a polymeric matrix, and (ii) microencapsulated drug, wherein the delay layer is substantially free of non-microencapsulated drug; and a second layer comprising (iii) a polymeric matrix, and (iv) non-microencapsulated drug matrix; wherein the second layer is located adjacent to the delay layer.

The difficulties with the above mentioned dosage forms are being of low cost effectiveness, requiring very special and expensive equipment and resulting in relatively small production of final dosage form.

Accordingly, there remains a need to provide alternative means of controlling delivery in a variety of patterns.

### SUMMARY OF THE INVENTION

In a first aspect, the present invention provides a dosage form for sustained release of paliperidone. The dosage form comprises at least a first component and second component, wherein the first component comprises at least one delay layer comprising a polymer, and the second component comprises non-coated Paliperidone and optionally comprises also coated Paliperidone; wherein the second component is located adjacent to the first component.

The dosage form of the first aspect may be coated with one or more additional delayed release layers, in the presence of Paliperidone, or in its absence.

In one of the aspects, the present invention provides methods for the preparation of the dosage forms described above.

In another aspect, the invention provides an extended release tablet of Paliperidone in the form of an inlay tablet. The inlay tablet comprises at least an inlay core and outer layer: (a) the inlay core comprising non-coated Paliperidone and at least one polymer capable of delaying the release of Paliperidone from the inlay core and capable of swelling upon hydration, wherein the inlay core optionally further comprises coated Paliperidone; and (b) the outer layer comprising a pharmaceutical excipient which is substantially water insoluble, wherein the outer layer partially surrounds the inlay core.

The invention provides a dosage form for the sustained release of Paliperidone, wherein the dosage form exhibits relative bioavailability, based on the area under the plasma concentration curve (AUC) for the same duration after oral administration in human subjects, of between about 1.5 and about 3.0, preferably between about 1.7 and about 3.0, and more preferably between about 1.9 and about 3.0, compared with commercially available INVEGA® extended release tablets containing the same amount of Paliperidone administered at the same dose in the human subjects.

The invention also provides a dosage form for the sustained release of Paliperidone, wherein the dosage form exhibits a relative Cmax, based on the plasma concentrations at various time after oral administration in human subjects, of between about 1.6 and about 3.0, preferably between about 1.7 and about 3.0, and more preferably between about 2.0 and about 3.0, compared with commercially available INVEGA® extended release tablets containing the same amount of Paliperidone, administered at the same dose in the human subjects.

In another aspect, the invention provides a process of making the inlay tablet of the invention, wherein the process comprises

(1) mixing Paliperidone and at least one polymer capable of delaying the release of Paliperidone and capable of swelling upon hydration, wherein at least part of the Paliperidone remains non-coated; and

(2) partially covering the mixture of step (1) with an outer layer comprising a pharmaceutical excipient which is substantially water insoluble to obtain the inlay tablet.

### BRIEF DESCRIPTION OF THE DRAWINGS

FIG. 1 is a schematic diagram showing an embodiment of the inlay tablet of the present invention, comprising an inlay core containing non-coated Paliperidone partially surrounded or incompletely covered by an inert insoluble outer layer.

FIG. 2 shows the dissolution profiles of the Paliperidone extended release tablets in the form of inlay tablets prepared according to Example 8.

FIG. 3 shows the least-square mean plasma concentrations versus time after the oral administration of the 6 mg inlay tablets of the present invention in the Test Group and the oral administration of the INVEGA 6 mg commercially available Paliperidone tablets in the Reference Group.

FIG. 4 shows the natural logarithm of the least-square mean plasma concentrations versus time after the oral administration of the 6 mg inlay tablets of the present invention in the Test Group and the oral administration of the INVEGA 6 mg commercially available Paliperidone tablets in the Reference Group.

### DETAILED DESCRIPTION OF THE INVENTION

In the present invention, the term “coated Paliperidone” means one or more Paliperidone particles which have been microencapsulated with at least one microencapsulation material. Such materials include, but are not limited to, proteins, polysaccharides, starches, waxes, fats, natural and synthetic polymers, and resins and/or combinations thereof. In this invention, “non-coated Paliperidone” means one or more Paliperidone particles which have not been microencapsulated with any microencapsulation material.

The term “microencapsulated with at least one microencapsulating material” means that the Paliperidone particles are surrounded by a layer of the at least one microencapsulating material without any pharmaceutical excipients existing between the Paliperidone particles and the layer of the at least one microencapsulating material.

In the present invention, the term “polymer” comprises natural and/or synthetic polymers and can also mean a combination of polymers of various types. The “polymer having an effect of delaying Paliperidone release” or “polymer capable of delaying the release of Paliperidone” includes polymers that form a viscous and gelatinous surface barrier or gel layer upon hydration, which barrier or gel layer controls Paliperidone release from and the penetration of liquids into the center of Paliperidone particles. The physicochemical characteristics of this barrier or gel layer control water uptake and the mechanism of Paliperidone release from the Paliperidone particles. The release of Paliperidone can occur via diffusion of Paliperidone through the barrier or gel layer, and preferably via gradual erosion of the barrier or gel layer. Suitable examples of the “polymer having an effect of delaying Paliperidone release” or “polymer capable of delaying the release of Paliperidone” include polyvinylpyrrolidone, polyethylene oxide such as POLYOX WSR-301, polysaccharides and hydrophilic cellulose derivatives such as methyl cellulose, hydroxypropyl methylcellulose, hydroxyethyl cellulose, hydroxypropyl cellulose, hydroxyethyl methylcellulose, carboxy methylcellulose and sodium carboxy methylcellulose. Preferred examples of the “polymer having an effect of delaying Paliperidone release” or “polymer capable of delaying the release of Paliperidone” include POLYOX WSR-301 and hydroxypropyl methylcellulose such as METHOCEL K15MP, K15M, K100M, K100LV, F4M, E4M, E3, E5, E10M, E15LV, E15LN, E15CLV, E50 and K3. Preferred examples of the polymer include METHOCEL K15MP and POLYOX WSR-301.

The term “delay layer” means a layer that functions, at least in part, to retard the release of the drug from the dosage form, including halting the release for a certain period of time.

In the invention, when two components (or two layers) are “adjacent”, that means the two components (or two layers) are in physical proximity with each other. Preferably, the two components (or two layers) are in direct contact at least one point.

In the invention, the term “core” refers to a component that is at least partially surrounded or covered by another component.

As used herein, the term “ascending release kinetics” means that the amount of Paliperidone released as a function of time increases over a period of time. Preferably, the amount of Paliperidone released as a function of time increases continuously, gradually and/or steadily, instead of in a step-wise fashion.

The first aspect of the present invention provides a dosage form for sustained release of paliperidone. The dosage form comprises at least two components, wherein the first component comprises at least one delay layer comprising a polymer, and the second component comprises non-coated Paliperidone; wherein the second component is located adjacent to the first component.

In an embodiment of the dosage form of the first aspect of the invention, the at least one delay layer of the first component may further comprise Paliperidone, but at least part of the Paliperidone is non-coated Paliperidone, so that the at least one delay layer does not further comprise only Paliperidone which is coated.

In one of the embodiments of the first aspect of the present invention, the sustained release Paliperidone dosage form comprises a plurality of particulates, wherein each of the particulates comprises at least two components: the first component comprising at least one delay layer comprising a polymer, and the second component comprising non-coated Paliperidone, wherein the second component is located adjacent to the first component.

Optionally, the second component further comprises coated Paliperidone.

According to an alternative embodiment of the preferred embodiment of the first aspect of the present invention, a layer of the first component contacts a layer of the second component along one face. Optionally, a layer of the second component is partially or completely surrounded by a layer of the first component. Optionally, the dosage form is partially or completely surrounded by a coating layer.

According to another alternative embodiment of the preferred embodiment of the first aspect of the present invention, the dosage form comprises three layers, so that two layers of the first component surround a layer of the second component. Optionally, at least one of the two layers of the first component also comprises non-coated Paliperidone. Optionally, the dosage form is partially or completely surrounded by a coating layer. The coating layer may also include non-coated Paliperidone.

According to an embodiment of the first aspect of the invention, the dosage form further comprises a sub-layer between a layer of the first component and a layer of the second component.

Each layer can contain other pharmaceutical excipients, so as to give suitable properties for compression, lubrication and/or binding as is well known to one skilled in the art.

In a second aspect, the present invention also discloses a multi-particulate dosage form, comprising a plurality of the particulates wherein each particulate comprises at least two components, wherein the first component comprises at least one extended release layer comprising a polymer and the second component comprises non-coated Paliperidone and optionally comprises also coated Paliperidone, wherein the second component is located adjacent to the first component.

Further said particulate may be covered by a delayed release layer.

According to a preferred embodiment of the second aspect of the present invention, said dosage form comprises at least a first population of particulates and at least a second population of the particulates, wherein said first population differs from said second population in at least one of 1) weight ratio between said first component and said second component; 2) weight ratio between coated and non-coated paliperidone, 3) weight ratio between paliperidone and other components in the particulate; 4) nature and thickness of coating layer; 5) existence and relative weight of core and/or sub layer; 6) existence of a second delay layer and the weight ratio between the layers.

In the aspects of the present invention described herein, the extended release layer preferably comprises at least one polymer such as hydroxypropyl cellulose, hydroxypropyl methyl cellulose, ethyl cellulose or polymethacrylates and at least one plasticizer. Preferred examples of the polymer in the extended release layer are METHOCEL K15MP AND ETHOCEL 7 CPS. The plasticizer can be hydrophilic such as triethyl citrate and polyethylene glycol and/or hydrophobic such as diethyl phthalate, dibutyl phthalate, dibutyl sebacate and acetyl tributyl citrate.

The cores employed in the second aspect of the invention described herein may be commercially available inert cores, such as microcrystalline cellulose spheres (e.g. CELLETS®), sugar spheres, or glass spheres. The cores employed in the second aspect of the invention described herein are covered by a layer comprising Paliperidone or a salt thereof. This layer preferably comprises Paliperidone or a salt thereof and at least one pharmaceutically acceptable excipient that acts as a binder.

The particulates in said dosage form may differ in the onset time of paliperidone release and in the rate of release after said onset time. According to a preferred embodiment of the present invention, an ascending release is achieved via controlling the parameters of composition and of relative location of the dosage-form layers.

According to an embodiment of the invention, those parameters are selected so that the onset time for liberation from the first population is earlier than that from the second population, etc.

In a third aspect, this invention also discloses a multi-particulate dosage form, comprising a plurality of the particulates wherein each particulates comprises at least three layers, wherein a first layer comprising non-coated Paliperidone or salt thereof and a polymer, a second layer is a delay release layer, which covers said the first Paliperidone layer and a third layer comprising non-coated Paliperidone together with a polymer.

According to a preferred embodiment of the third aspect of the invention, the second layer is a pH-sensitive layer, and the third layer is a delayed release layer that comprises said non-coated Paliperidone. This dosage form may comprise at least one additional delayed release layer that covers the first layer.

The core employed in the third aspect of the invention described herein may be one of the commercially available inert cores, such as microcrystalline cellulose spheres (e.g. CELLETS®), sugar spheres, or glass spheres. The core employed in the third aspect of the invention described herein is covered by a layer comprising Paliperidone or a salt thereof. This layer preferably comprises Paliperidone or a salt thereof and at least one pharmaceutically acceptable excipient that acts as a binder such as methyl cellulose, hydroxypropyl cellulose, hydroxypropyl methyl cellulose and povidone, copovidone, starch, Arabic gum, acasia gum, gelatin.

According to a preferred embodiment of the third aspect of the invention, a substantial fraction of the Paliperidone in the third layer is released before the release of a fraction of Paliperidone in the first layer. By adding an organic acid to the layer coating the pH sensitive layer, a micro pH acidic environment can be obtained, so that the pH sensitive delay layer cannot dissolve until the acid comprising layer is released from the dosage form completely. By this way the release of the inner Paliperidone layer can be controlled.

The term “sustained release dosage form” means a dosage form that releases the drug for 4-24 hours. The dosage forms in accord with the present invention exhibit t90 values of at least 4 hours or more and preferably up to about 24 hours or more, for once per daily dosing. The dosage forms continuously release drug for sustained periods of at least about 6 hours, preferably about 8 hours or more and, in particular embodiments, about 12 hours or more.

The fourth aspect of the invention is directed to Paliperidone extended release tablets in the form of inlay tablets. The inlay tablet comprises at least an inlay core and outer layer: (a) the inlay core comprising non-coated Paliperidone and at least one polymer capable of delaying the release of Paliperidone from the inlay core and capable of swelling upon hydration, wherein the inlay core optionally can further comprise coated Paliperidone; and (b) the outer layer comprising a pharmaceutical excipient which is substantially water insoluble, wherein the outer layer partially surrounds the inlay core.

The at least one “polymer capable of delaying the release of Paliperidone from the inlay core and capable of swelling upon hydration” is at least one polymer that forms a viscous and gelatinous surface barrier or gel layer upon hydration, which barrier or gel layer controls Paliperidone release from and the penetration of liquids into Paliperidone particles. The release of Paliperidone can occur via diffusion of Paliperidone through the barrier or gel layer, and/or via gradual erosion of the barrier or gel layer. Suitable examples of the polymer include polyvinylpyrrolidone, poly(ethylene oxide) such as POLYOX WSR-301, polysaccharides and hydrophilic cellulose derivatives such as methyl cellulose, ethyl cellulose, hydroxypropyl methylcellulose, hydroxyethyl cellulose, hydroxypropyl cellulose, hydroxyethyl methylcellulose, carboxy methylcellulose and sodium carboxy methylcellulose. Preferred examples of the “polymer having an effect of delaying Paliperidone release from the particles” include POLYOX WSR-301 and hydroxypropyl methylcellulose such as METHOCEL K15MP, K15M, K100M, K100LV, F4M, E4M, E3, E5, E15M, E15LV, E15LN, E15CLV, E50 and K3. A preferred at least one “polymer capable of delaying the release of Paliperidone from the inlay core and capable of swelling upon hydration” is POLYOX WSR-301.

The “pharmaceutical excipient which is substantially water insoluble” of the outer layer renders the outer layer substantially water insoluble. Examples of “pharmaceutical excipient which is substantially water insoluble” in the outer layer include pharmaceutically acceptable polymers which are substantially water insoluble, such as pharmaceutically acceptable drug release modifying polymers which are substantially water insoluble. Suitable pharmaceutically acceptable polymers which are substantially water insoluble include cationic copolymers of ethylacrylate and methylacrylate with quarternary ammonium groups such as EUDRAGIT RS and EUDRAGIT RL, ethylacrylate methylmethacrylate copolymer with neutral ester groups, cellulose esters, cellulose ethers and cellulose esterethers, ethyl cellulose such as ETHYL CELLULOSE T10 PHARM, cellulose acetate, cellulose diacetate, cellulose triacetate and polyester polymers. Non-limiting examples of the polyester polymers that can be used include poly(ε-caprolactone)s, poly(alkylene glycol adipate)s such as poly(ethylene glycol adipate), poly(propylene glycol adipate) and poly(butylene glycol adipate), polyvinyl acetate and blends and copolymers thereof. A preferred “pharmaceutical excipient which is substantially water insoluble” is ETHYLCELLULOSE T10 PHARM.

The inlay core preferably is in the form of a tablet or compressed slug.

The inlay core and the outer layer independently can further comprise at least one other pharmaceutical excipient such as pharmaceutically acceptable fillers, diluents, pH modifiers, glidants, lubricants, binders, dyes and flavoring agents.

In some of the embodiments of the inlay tablet of the invention, the inlay core comprises about 1-3% w/w Paliperidone, about 2-5% w/w filler such as STARLAC, about 5-15% w/w pH modifier such as magnesium carbonate, about 5-20% w/w release modifying polymer such as POLOYX WSR-301, about 0-1% w/w lubricant such as stearic acid and about 0-1% w/w glidant such as silicon dioxide.

In some of the embodiments of the inlay tablet of the invention, the outer layer comprises about 50-90% w/w release modifying polymer such as ETHYLCELLULOSE T10 PHARM, about 0-1% w/w dye such as FERRIC OXIDE YELLOW NF and about 0-1% w/w lubricant such as stearic acid.

Another aspect of the invention is directed to a dosage form for the sustained release of Paliperidone, wherein the dosage form exhibits relative bioavailability, based on the area under the plasma concentration curve (AUC) for the same duration, e.g., 0 to 96 hours, or 0 hour to infinity, after oral administration, of at least about 1.5, preferably at least about 1.7, and more preferably at least about 1.9, compared with commercially available INVEGA® extended release tablets containing the same amount of Paliperidone administered at the same dose. By a relative bioavailability, based on AUC, of at least about 1.5, it means that the AUC achieved in the human subjects orally administered the dosage form according to the first aspect of the invention is at least about 50% higher than the AUC, for the same duration, achieved in the human subjects orally administered the commercially available INVEGA® extended release tablets containing the same amount of Paliperidone, wherein the Paliperidone is administered at the same dose.

In one of the embodiments, the invention provides a dosage form for the sustained release of Paliperidone, wherein the dosage form exhibits relative bioavailability, based on the area under the plasma concentration curve (AUC) for the same duration after oral administration, of between about 1.5 and about 3.0, preferably between about 1.7 and about 3.0, and more preferably between about 1.9 and about 3.0, compared with commercially available INVEGA® extended release tablets containing the same amount of Paliperidone administered at the same dose.

Another aspect of the invention is directed to a dosage form for the sustained release of Paliperidone, wherein the dosage form exhibits a relative Cmax, based on the plasma concentrations at various time after oral administration in human subjects, of at least about 1.6, preferably at least about 1.7, and more preferably at least about 2.0, compared with commercially available INVEGA® extended release tablets containing the same amount of Paliperidone, administered at the same dose in the human subjects.

In one of the embodiments, the invention also provides a dosage form for the sustained release of Paliperidone, wherein the dosage form exhibits a relative Cmax, based on the plasma concentrations at various time after oral administration in human subjects, of between about 1.6 and about 3.0, preferably between about 1.7 and about 3.0, and more preferably between about 2.0 and about 3.0, compared with commercially available INVEGA® extended release tablets containing the same amount of Paliperidone, administered at the same dose in the human subjects.

Another aspect of the invention is directed to a dosage form for the sustained release of Paliperidone, wherein the dosage form exhibits an in vitro dissolution profile determined using a 50 RPM paddle method in a dissolution medium of 500 ml 0.05 M phosphate buffer, pH 6.8, 37° C., wherein the dissolution profile is less than about 10% dissolution in 4 hours, between about 10% to about 25% dissolution in 8 hours, between about 40% to about 60% dissolution in 16 hours and not less than about 70% in 24 hours after the start of the dissolution study, respectively.

In the fifth aspect, the invention provides a process of making the inlay tablet of the invention, wherein the process comprises

(1) mixing Paliperidone and at least one polymer capable of delaying the release of Paliperidone and capable of swelling upon hydration; and

(2) partially covering the mixture of step (1) with an outer layer comprising a pharmaceutical excipient which is substantially water insoluble to obtain the inlay tablet.

In a preferred embodiment of the process of preparing the inlay tablet, the mixture of step (1) is compressed into a slug or tablet before step (2).

In a further preferred embodiment of the process of preparing the inlay tablet, the mixture of step (1) is compressed into a slug or tablet before step (2); the slug or tablet is mixed with the at least one polymer capable of delaying the release of Paliperidone and capable of swelling upon hydration to form a mixture; and the mixture is compressed into a tablet before step (2). More preferably, a filler, pH modifier, glidant and/or lubricant is added in step (1). Optionally, the slug or tablet formed by compression is milled before being mixed with the at least one polymer capable of delaying the release of Paliperidone and capable of swelling upon hydration to form the mixture to be compressed into a tablet before step (2).

Preferably, in the process of preparing the inlay tablet, the product of step (2) is compressed to obtain the inlay tablet.

Some of the embodiments of the inlay tablets such as exemplified in Example 8 can be prepared according to the ingredients listed in the table below.

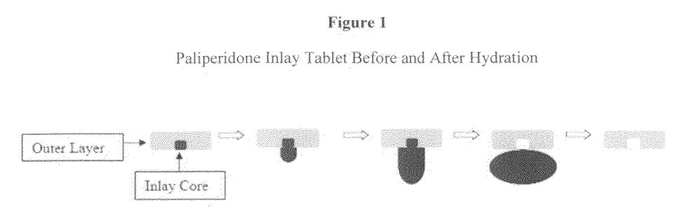

Without being bound to any hypothesis, it is believed that the inlay tablet may function in the extended release of Paliperidone according to a theory schematically illustrated in FIG. 1, which shows an embodiment of the inlay tablet of the present invention. The tablet drawn in the far left of FIG. 1 depicts the inlay tablet before exposure to an aqueous medium, and the four tablets drawn in the right depict four stages of the inlay tablet exposed to an aqueous medium for increasing length of time demonstrating gel formation in the inlay core and the gradual swelling of the gel of the inlay core due to the absorption of water by the inlay core through the surface not covered by the inert insoluble outer layer, resulting in a gradual increase of the free surface of the inlay core leading to an ascending release of Paliperidone from the inlay core.

In all aspects of the invention described herein, the pharmaceutical composition preferably further comprises one or more pharmaceutical excipients.

The term “pharmaceutical excipients” means any pharmaceutically acceptable substances, other than the active drug substance or finished dosage form, that have been appropriately evaluated for safety and are included in drug delivery systems to (a) help in the processing of the drug delivery system during its manufacture; (b) support, protect or enhance the stability and/or bioavailability of the active drug substance; (c) make the active drug substance or the final dosage form more acceptable by the patients; (d) enhance the overall safety, effectiveness or delivery of the active drug substance during storage or use; or (e) help in product identification. The pharmaceutical excipients are added to aid the formulation and manufacture of the final dosage form for administration to the patients. The pharmaceutical excipients can be mixed with the active drug substance to make the final dosage form. Examples of “pharmaceutical excipients” include pharmaceutical grade fillers, diluents, pH modifiers, release modifying polymers, lubricants, glidants, disintegrants, carriers, bulking agents, binders, wetting agents, dyes (e.g., ferric oxide yellow and iron oxide red) and flavoring agents. Other excipients that may be incorporated into the final dosage form include preservatives, surfactants, antioxidants and any other excipient commonly used in the pharmaceutical industry.

Suitable fillers and diluents include, but are not limited to, cellulose-derived materials like powdered cellulose, microcrystalline cellulose (e.g. Avicel®), microfine cellulose, methyl cellulose, ethyl cellulose, hydroxyethyl cellulose, hydroxypropyl cellulose (e.g. Klucel®), hydroxypropyl methylcellulose, carboxymethyl cellulose salts (such as carboxymethyl cellulose calcium) and other substituted and unsubstituted celluloses; starch such as maize starch; pregelatinized starch; lactose, preferably lactose monohydrate (e.g. Pharmatose®); talc; waxes; sugars; sugar alcohols like mannitol and sorbitol; acrylate polymers and copolymers; dextrates; dextrin; dextrose; maltodextrin; pectin; gelatin; inorganic diluents like calcium carbonate, dibasic calcium phosphate dihydrate, tribasic calcium phosphate, calcium sulfate, magnesium carbonate, magnesium oxide, sodium chloride, combine materials like STARLAC and other diluents known to the pharmaceutical industry. More preferred fillers include talc, lactose monohydrate, pregelatinized starch, mannitol or sorbitol. An even more preferred filler is STARLAC.

Suitable pH modifiers are pharmaceutically acceptable buffering compounds such as alkaline earth metal carbonates, alkali metal carbonates, alkaline earth metal bicarbonates, alkali metal bicarbonates and magnesium oxide, e.g., magnesium carbonate, calcium carbonate, magnesium bicarbonate, calcium bicarbonate, sodium carbonate, potassium carbonate, sodium bicarbonate and potassium bicarbonate. A preferred pH modifier is magnesium carbonate.

Suitable disintegrants include croscarmellose sodium (e.g. Ac Di Sol®, Primellose®), crospovidone (e.g. Kollidon®, Polyplasdone®), microcrystalline cellulose, polacrilin potassium, powdered cellulose, pregelatinized starch, sodium starch glycolate (e.g. Explotab®, Primoljel®) and starch. Preferred disintegrants include Copovidone and microcrystalline cellulose.

Glidants can be added to improve the flowability of a solid composition before compaction and to improve the accuracy of dosing especially during compaction and capsule filling. Excipients that may function as glidants include colloidal silicon dioxide, magnesium trisilicate, powdered cellulose, and talc. The preferred glidant is colloidal silicon dioxide.

A lubricant may be added to the pharmaceutical compositions of the present invention to reduce adhesion and/or ease the release of the product from e.g. the die. Suitable lubricants include, but are not limited to, stearic acid, magnesium stearate, calcium stearate, glyceryl monostearate, glyceryl palmitostearate, hydrogenated castor oil, hydrogenated vegetable oil, mineral oil, polyethylene glycol, sodium lauryl sulfate, sodium stearyl fumarate, stearic acid, talc and zinc stearate. Stearic acid and magnesium stearate are preferred.

Carriers include, but are not limited to, lactose, white sugar, sodium chloride, glucose, urea, starch, calcium carbonate, kaolin, crystalline cellulose, and silicic acid.

Binders include, but are not limited to, carboxymethyl cellulose, shelac, methyl cellulose, hydroxypropyl methylcellulose, HPMC, starch and polyvinylpyrrolidone. Other suitable binders include, but are not limited to, acacia gum, pregelatinized starch, sodium alginate, glucose and other binders used in wet and dry granulation and direct compression tableting processes.

Flavoring agents and flavor enhancers make the dosage form more palatable to the patient. Common flavoring agents and flavor enhancers for pharmaceutical products that can be included in the composition of the present invention include, but are not limited to, maltol, vanillin, ethyl vanillin, menthol, citric acid, fumaric acid, ethyl maltol, and tartaric acid.

According to the preferred embodiment, the total amount of Paliperidone in the dosage form ranges from about 1 mg to about 15 mg.

Some of the embodiments of the dosage forms of the invention do not contain coated Paliperidone.

Some of the embodiments of the dosage forms of the invention may contain coated Paliperidone.

In some of the embodiments of the dosage forms of the invention, the dosage forms may contain coated Paliperidone along with non-coated Paliperidone in one or more of the layers, or in one or more of the components.

In some of the embodiments of the dosage forms of the invention comprising a delayed release layer, the delayed release layer can further comprise Paliperidone. For instance, dosage forms having a delayed release layer further comprising Paliperidone are exemplified in Examples 1, 5 and 7 below.

Methods for making multilayered are described by W. C. Gunsel, Compression coated and layer tablets in Pharmaceutical Dosage Forms: Tablets, Vol 1, edited by H. H. Lieberman.

In a fourth aspect, the invention provides a process for preparing a pharmaceutical composition as described above in relation to the second aspect of the invention.

The process comprises preparing a sphere by coating a core with a layer comprising Paliperidone or a salt thereof and applying an extended release layer thereon.

The layer comprising Paliperidone or a salt thereof may be applied using any conventional method. Preferably, the core is coated with a Paliperidone containing layer using a solution/dispersion of Paliperidone or a salt thereof and a binder. The coating process is preferably performed using a fluidized bed coater, preferably equipped with a bottom or top spray device.

In another aspect of the invention described herein, the processes for preparing a final dosage composition of the invention preferably further comprises mixing a plurality of the spheres, which provide a sustained release of paliperidone or a salt thereof, with at least one pharmaceutically acceptable excipient such as a filler, binder, glidant, disintegrant or lubricant. The mixture can be filled into capsules or sachets or compressed into tablets.

The mixture may be compressed into tablets in the following way. The coated spheres are mixed with at least one pharmaceutically acceptable excipient and compressed into tablets. The excipients can be fillers such as microcrystalline cellulose, lactose monohydrate, maize starch, powdered cellulose, sorbitol and mannitol; binders such as povidone, hydroxypropyl cellulose, and hydroxypropyl methyl cellulose; glidants such as talc, and silicon dioxide; disintegrants such as croscarmellose sodium, pregelatinized starch, crospovidone, hydroxypropyl cellulose, and sodium starch glycolate; and lubricants such as stearic acid, magnesium stearate, mineral oil, hydrogenated castor oil and sodium stearyl fumarate.

### EXAMPLES

### Examples of Paliperidone Extended Release Tablets

### Example 1

### Paliperidone 9 mg Tablets with Ascending Release Kinetics

Layer 1 (Inner Core)

| Ingredient | mg/Tablet |
|---|---|
| Paliperidone | 6.0 |
| Lactose Monohydrate | 73.5 |
| Methocel K15MP | 20.0 |
| Magnesium Stearate | 0.5 |
| Total Weight | 100.0 |

Layer 2 (pH Dependent Coating)

| Ingredient | mg/Tablet |
|---|---|
| Eudragit FS 30D | 3.3 |
| Triethyl citrate | 0.4 |
| Talc | 1.3 |
| Water (process solvent) | |
| Total Weight | 5.0 |

Layer 3 (Outer Coating)

| Ingredient | mg/Tablet |
|---|---|
| Paliperidone | 3.0 |
| Lactose Monohydrate | 141.0 |
| Fumaric Acid | 10.0 |
| Methocel K15MP | 40.0 |
| Magnesium Stearate | 1.0 |
| Total Weight | 195.0 |
| Total Tablet Weight | 300.0 |

### Process

1. The inner tablet is prepared by mixing Paliperidone, Lactose Monohydrate, Methocel K15MP in a mixer, adding sieved Magnesium Stearate to the blend, mixing, and pressing the mixed blend to form tablets in a suitable tablet press.
2. The tablets from step 1 is coated with the coating dispersion to form Layer 2
3. Layer 3 is formed by mixing Paliperidone, Lactose Monohydrate, Methocel K15MP and Fumaric Acid in a mixer, adding sieved Magnesium Stearate to the blend and mixing.
4. The final tablets are prepared by press coating the tablets from step 2 with the blend from step 3.

### Example 2

### Paliperidone 9 mg Multi-Particulate Dosage Form

Pellets, Layer 1 (Active Core)

| Ingredient | mg/product |
|---|---|
| Paliperidone | 9.0 |
| Microcrystalline Cellulose | 91.0 |
| Water (process solvent) | |
| Total Weight | 100.0 |

Layer 2

| Ingredient | mg/layer |
|---|---|
| Ethocel 7 CPS | 20.0 |
| PEG 3350 | 5.0 |
| Alcohol 95% (process solvent) | |
| Isopropyl Alcohol (process solvent) | |
| Water (process solvent) | |
| Total Weight | 25.0 |

### Process

1. The core is prepared by mixing Paliperidone and Microcrystalline Cellulose in a mixer, adding water to the blend and mixing. Using extrusion and spheronization technique, pellets with diameter 200-1000 mm are produced and dried in a suitable dryer.
2. Ethocel 7 CPS and PEG 3350 are dissolved in a mixture of the process solvents.
3. The core is coated with the solution from step 2.

### Example 3

### Paliperidone 9 mg Multi-Particulate Dosage Form

Pellets, Layer 1 (Active Core))

| Ingredient | mg/product |
|---|---|
| Paliperidone | 9.0 |
| Microcrystalline Cellulose | 91 |
| Water (process solvent) | |
| Total Weight | 100.0 |

Layer 2

| Ingredient | mg/product |
|---|---|
| Ethocel 7 CPS | 20.0 |
| PEG 3350 | 15.0 |
| Alcohol 95% (process solvent) | |
| Isopropyl Alcohol (process solvent) | |
| Water (process solvent) | |
| Total Weight | 35.0 |

Layer 3 (DR Coating)

| Ingredient | mg/product |
|---|---|
| Methocel K15MP | 30.0 |
| PEG 3350 | 10.0 |
| Talc | 5.0 |
| Ethanol (process solvent) | |
| Water (process solvent) | |
| Total Weight | 45.0 |

{Inventors: What does “DR” stand for?}

### Process

1. The core is made by mixing Paliperidone and Microcrystalline Cellulose in a mixer, adding water to the blend and mixing. Using extrusion & spheronization technique, pellets with diameter 200-1000 mm are produced and dried in a suitable dryer.
2. Ethocel 7 CPS and PEG 3350 are dissolved in a mixture of the process solvents.
3. The core is coated with the solution from step 2.
4. Methocel K15MP and PEG 3350 were dissolved in a mixture of the process solvents. Talc was added and mixed until dispersed.
5. The pellets were coated with the dispersion from step 4.

### Example 4

### Paliperidone 9 mg Multi-Particulate Dosage Form

A 1:1.33 (w/w) mixture of two types of pellets prepared as described in Examples 2 and 3 is prepared with ascending release kinetics

### Example 5

### Paliperidone 9 mg Multi-Particulate Dosage Form

Pellets, Layer 1 (First Drug Layer)

| Ingredient | mg/product |
|---|---|
| sugar spheres | 90.0 |
| Paliperidone | 6.0 |
| Copovidone | 4.0 |
| Water (process solvent) | |
| Total Weight | 100.0 |

Layer 2

| Ingredient | mg/product |
|---|---|
| Ethocel 7 CPS | 20.0 |
| PEG 3350 | 15.0 |
| Alcohol 95% (process solvent) | |
| Isopropyl Alcohol (process solvent) | |
| Water (process solvent) | |
| Total Weight | 35.0 |

Layer 3 (Second Drug Layer)

| Ingredient | mg/product |
|---|---|
| Paliperidone | 3.0 |
| Methocel K15MP | 20.0 |
| PEG 3350 | 7.0 |
| Talc | 6.0 |
| Ethanol (process solvent) | |
| Water (process solvent) | |
| Total Weight | 33.0 |

### Process

1. The first drug layer is made by coating the sugar spheres with the dispersion of Paliperidone in a Copovidone aqueous solution.
2. Ethocel 7 CPS and PEG 3350 are dissolved in a mixture of the process solvents.
3. The pellets are coated with the solution from step 2
4. Methocel K15MP and PEG 3350 are dissolved in a mixture of the process solvents. Talc and Paliperidone are added and mixed until dispersed.
5. The second drug layer is made by coating the pellets with the dispersion from step 4.

### Example 6

### Paliperidone 9 mg Tablets with Multi-Particulate Dosage Form, with ascending release kinetics)

### Process

1. The pellets from Example 5 are mixed with Lactose SD (2:1 w/w), 1% of magnesium stearate is added and mixed.
2. The tablets are compressed on a suitable tablet press.

### Example 7

### Paliperidone 9 mg Multi-Particulate Dosage Form

Pellets, Layer 1 (First Drug Layer)

| Ingredient | mg/product |
|---|---|
| cellulose spheres | 90.0 |
| Paliperidone | 6.0 |
| Copovidone | 4.0 |
| Water (process solvent) | |
| Total Weight | 100.0 |

Layer 2 (DR coating)

| Ingredient | mg/product |
|---|---|
| Ethocel 7 CPS | 20.0 |
| PEG 3350 | 15.0 |
| Alcohol 95% (process solvent) | |
| Isopropyl Alcohol (process solvent) | |
| Water (process solvent) | |
| Total Weight | 35.0 |

Layer 3 (pH Dependent Coating)

| Ingredient | mg/product |
|---|---|
| Eudragit FS 30D | 8.0 |
| Triethyl citrate | 1.2 |
| Talc | 1.3 |
| Water (process solvent) | |
| Total Weight | 10.5 |

Layer 4 (Second Drug Layer)

| Ingredient | mg/product |
|---|---|
| Paliperidone | 3.0 |
| Methocel K15MP | 20.0 |
| PEG 3350 | 7.0 |
| Fumaric Acid | 10.0 |
| Talc | 6.0 |
| Ethanol (process solvent) | |
| Water (process solvent) | |
| Total Weight | 46.0 |

### Process

1. The first drug layer is made by coating the sugar spheres with the dispersion of Paliperidone in a Copovidone aqueous solution.
2. Ethocel 7 CPS and PEG 3350 are dissolved in a mixture of the process solvents.
3. The ER layer is prepared by coating the pellets with the solution from step 2.
4. The pH control layer is prepared by coating the pellets with the coating dispersion from Layer 3
5. Methocel K15MP and PEG 3350 are dissolved in a mixture of the process solvents. Talc and Paliperidone are added and mixed until dispersed.
6. The second drug ER layer is prepared by coating the pellets with the dispersion from step 5

### Example 8

### Paliperidone Extended Release Tablets in the Form of Inlay Tablets

Formulation of a Paliperidone Inlay Tablet

| | % | | |
|---|---|---|---|
| | W/W | mg per | |
| MATERIAL | range | tablet | FUNCTION |
| Inlay Cores | | | |
| PALIPERIDONE | 1-3 | 6.0 | API |
| STARLAC | 2-5 | 12.0 | Filler |
| MAGNESIUM CARBONATE USP | 5-15 | 26.0 | pH modifier |
| POLYOX WSR-301 | 5-20 | 40.0 | Release |
| | | | modifying |
| | | | polymer |
| STEARIC ACID NF/EP | 0-1 | 1.5 | Lubricant |
| Silicone Dioxide NF | 0-1 | 0.5 | Glidant |
| (Syloid 244 FP) | | | |
| Outer Layer | | | |
| ETHYLCELLULOSE | 50-90 | 216.5 | Release |
| T10 PHARM | | | modifying |
| | | | polymer |
| FERRIC OXIDE YELLOW NF | 0-1 | 0.5 | Dye |
| STEARIC ACID NF/EP | 0-1 | 2.0 | Lubricant |
| Final Inlay Tablet | | 305.0 | |

Paliperidone extended release tablets in the form of inlay tablets were prepared with the following process.

### Process

1.90 g Paliperidone, 180 g STARLAC, 390 g magnesium carbonate, 75 g POLYOX WSR-301 and 7.5 g silicone dioxide were sieved and mixed in a V-blender.

2. 15 g Stearic acid was sieved and added to the blend from step 1 and mixed.

3. The blend from step 2 was then compressed into slugs using a 20 mm flat punch.

4. The slugs were milled using a 0.8 mm screen.

5. 378.75 g of Milled slugs were then mixed with 272.5 g POLYOX WSR-301 and 3.75 g sieved stearic acid in a V-blender.

6. The blend from step 5 was then compressed into 86 mg tablets using a 5.5 mm normal concave punch, wherein the 86 mg tablets were used as the inlay cores in the rest of the process.

7. 3247.5 g of ETHYLCELLULOSE T10 PHARM and 7.5 g yellow ferric oxide were sieved and mixed in a V-blender.

8. 30 g Stearic acid was sieved and added to the blend from step 7 and mixed.

9. The tablets from step 6 were then recompressed with the blend from step 8 to create an incomplete outer layer on the tablets from step 6 in order to form inlay tablets, each weighing 305 mg, as the Paliperidone extended release tablets, wherein each of the tablets from step 6 acts as the inlay core for the Paliperidone extended release tablets.

### Example 9

### Dissolution Profile of the Inlay Tablets

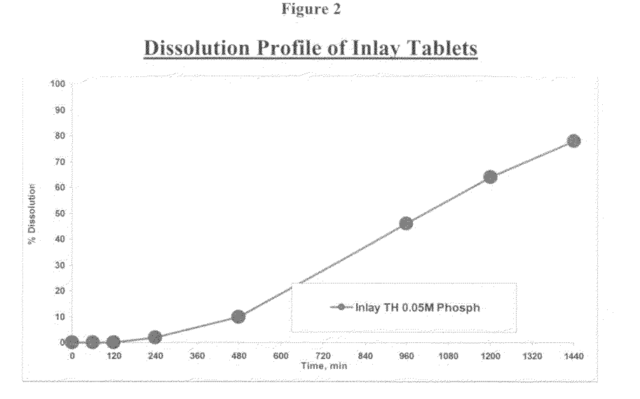

The dissolution of Paliperidone in the Paliperidone extended release tablets prepared as described in Example 8 were determined using a 50 RPM paddle method in a dissolution medium of 500 ml 0.05 M phosphate buffer, pH 6.8, wherein the dissolution was measured from 0 to 1440 minutes, i.e., 0 to 24 hours. The dissolution data are shown in FIG. 2.

### Example 10

### Pharmacokinetics of the Inlay Tablets

The pharmacokinetics of the Paliperidone extended release tablets, in the form of the inlay tablets containing 6 mg Paliperidone, of the present invention were determined in a group of 16 human subjects orally administered the inlay tablets. For comparison purposes, the pharmacokinetics of commercially available Paliperidone tablets, INVEGA 6 mg, were also determined in the 16 human subjects after oral administration. Plasma concentrations of Paliperidone in the 16 human subjects were measured at 0 to 96 hours after oral administration of the Paliperidone extended release tablets of the present invention (i.e., the Test Group) or after oral administration of the commercially available INVEGA 6 mg Paliperidone tablets (i.e., the Reference Group).

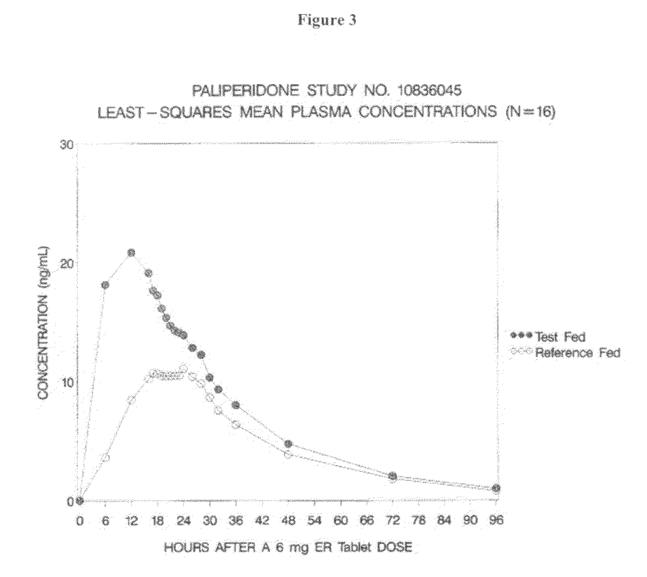

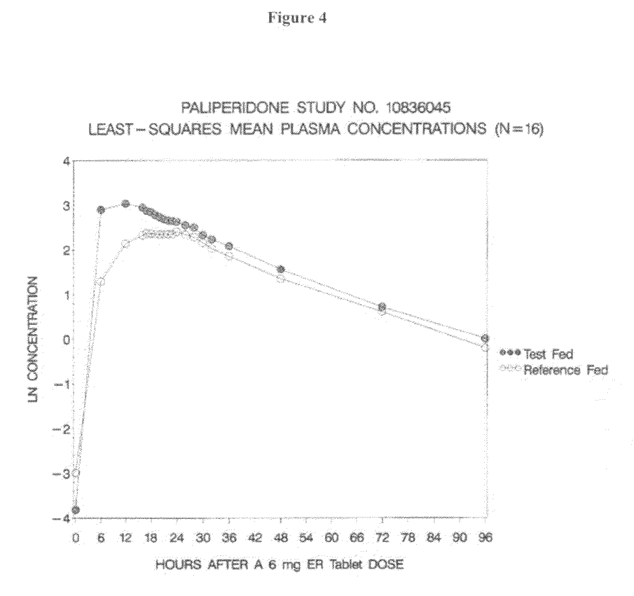

The least-square mean plasma concentrations versus time after the oral administration of the Test Group and Reference Group are shown in FIG. 3. The natural log of the least-square mean plasma concentrations versus time after the oral administration of the Test Group and Reference Group are shown in FIG. 4. The mean values of the pharmacokinetic (PK) parameters of the Test Group and Reference Group are shown in the table below.

Mean PK Parameter Values

| PK Parameters | Test Group | Reference Group |
|---|---|---|
| AUC_{96 h} (h · ng/ml) | 702.13 | 435.94 |
| AUC_{0-∞} (h · ng/ml) | 733.43 | 461.10 |
| Cₘₐₓ (ng/ml) | 22.33 | 12.82 |
| T_{1/2} (h) | 20.17 | 19.96 |
| kₑ | 0.04 | 0.04 |
| LN AUC_{96 h} | 632.38 | 357.42 |
| LN AUC_{0-∞} | 659.11 | 375.70 |
| LN Cₘₐₓ | 20.33 | 10.59 |

The ratio of the mean values of the pharmacokinetic (PK) parameters of the Test Group and Reference Group, as well as the 90% confidence intervals (CI) are shown in the table below.

Ratios of Mean PK Parameter Values
(Test Group/Reference Group)

| | | | | Treatment |
|---|---|---|---|---|
| PK Parameters | Ratio | Lower CI | Higher CI | Effect (p) |
| AUC_{96 h} | 1.6106 | 1.2223 | 1.9990 | 0.0151 |
| AUC_{0-∞} | 1.5906 | 1.2012 | 1.9800 | 0.0182 |
| Cₘₐₓ | 1.7416 | 1.3451 | 2.1381 | 0.0053 |
| T_{1/2} | 1.0109 | 0.9617 | 1.0600 | |
| kₑ | 1.0041 | 0.9606 | 1.0477 | |
| LN AUC_{96 h} | 1.7693 | 1.4459 | 2.1651 | 0.0002 |
| LN AUC_{0-∞} | 1.7544 | 1.4356 | 2.1439 | 0.0002 |
| LN Cₘₐₓ | 1.9192 | 1.5686 | 2.3481 | 0.0001 |

### Example 11

### Paliperidone 3 mg Tablets with Ascending Release Kinetics

Layer 1 (external layer)

| Ingredient | mg/Tablet |
|---|---|
| Polyethylene oxide (Polyox WSR-301) | 95.00 |
| Microcrystalline Cellulose (Avicel PH 101) | 28.75 |
| Magnesium Stearate | 1.25 |
| Total Weight | 125.00 |

Layer 2 (internal layer)

| Ingredient | mg/Tablet |
|---|---|
| Paliperidone | 3.0 |
| Polyethylene oxide (Polyox WSR-301) | 60.0 |
| Microcrystalline Cellulose (Avicel PH 101) | 16.0 |
| Sodium Chloride | 20.0 |
| Magnesium Stearate | 1.0 |
| Total Weight | 100.0 |

Layer 3 (external layer)

| Ingredient | mg/Tablet |
|---|---|
| Polyethylene oxide (Polyox WSR-301) | 95.00 |
| Microcrystalline Cellulose (Avicel PH 101) | 28.75 |
| Magnesium Stearate | 1.25 |
| Total Weight | 125.00 |
| Total Tablet Weight | 350.0 |

### Process

1. The composition of external layers is prepared by mixing polyethylene oxide (POLYOX WSR-301), microcrystalline cellulose (Avicel PH 101) in a mixer, adding sieved magnesium stearate to the blend and mixing.
2. The composition of internal layer is prepared by mixing Paliperidone, polyethylene oxide (POLYOX WSR-301), microcrystalline cellulose (Avicel PH 101) and sodium chloride in a mixer, adding sieved magnesium stearate to the blend and mixing.
3. The tablets are prepared by pressing the mixed blends to form three-layer tablets in a suitable tablet press, wherein two sides of the internal layer are in contact with the two external layers

### Example 12

### Paliperidone 6 mg Tablets with Ascending Release Kinetics

Layer 1 (external layer)

| Ingredient | mg/Tablet |
|---|---|
| Hydroxypropyl methylcellulose (Methocel E10M) | 65.0 |
| Ethylcellulose T10 | 60.0 |
| Povidone K-90 | 3.0 |
| Stearic Acid | 2.0 |
| Water - process solvent | |
| Total Weight | 130.00 |

Layer 2 (internal layer)

| Ingredient | mg/Tablet |
|---|---|
| Paliperidone | 6.0 |
| Hydroxypropyl methylcellulose (Methocel E10M) | 60.0 |
| Ethylcellulose T10 | 50.0 |
| Povidone K-90 | 2.0 |
| Stearic Acid | 2.0 |
| Water - process solvent | |
| Total Weight | 120.0 |

Layer 3 (external layer)

| Ingredient | mg/Tablet |
|---|---|
| Hydroxypropyl methylcellulose (Methocel E10M) | 65.0 |
| Ethylcellulose T10 | 60.0 |
| Povidone K-90 | 3.0 |
| Stearic Acid | 2.0 |
| Water - process solvent | |
| Total Weight | 130.00 |
| Total Tablet Weight | 380.0 |

### Process

1. The composition of external layers is prepared by granulation of hydroxypropyl methylcellulose (Methocel E10M), ETHYLCELLULOSE T10 and Povidone K-90 in a high-shear mixer using water as a process solvent, drying and milling of the granulate, adding sieved Stearic Acid to the milled granulate and mixing.
2. The composition of internal layer is prepared by granulation OF Paliperidone,
Hydroxypropyl methylcellulose (Methocel E10M), ETHYLCELLULOSE T10 and Povidone K-90 in a high-shear mixer using water as a process solvent, drying and milling of the granulate, adding sieved stearic acid to the milled granulate and mixing.
   3. The tablets are prepared by pressing the mixed blends to form three-layer tablets in a suitable tablet press, when two sides of the internal layer are in contact with the two external layers.

We claim:

1. A dosage form for sustained release of Paliperidone, comprising at least a first component and a second component located adjacent to the first component, wherein
the first component comprises at least one delay layer comprising a polymer, and the second component comprises non-coated Paliperidone.

2. The dosage form of claim 1, wherein the second component further comprises coated Paliperidone.

3. The dosage form of claim 1, further comprising a coating.

4. The dosage form of claim 1, wherein the first component further comprises non-coated Paliperidone.

5. The dosage form of claim 1, wherein (i) a layer of the first component contacts a layer of the second component along one face; (ii) the dosage form comprises three layers, so that two layers of the first component surround a layer of the second component; (iii) a layer of the second component is partially or completely surrounded by a layer of the first component; (iv) a layer of the second component is partially or completely surrounded by a sub-layer, which is partially or completely surrounded by a layer of the first component or (v) any one of (i) to (iv) further partially or completely surrounded by a coating layer.

6. The dosage form of claim 1, further comprising a sub-layer between the layer of the first component and the layer of the second component.

7. The dosage form of claim 1 exhibiting relative bioavailability, based on the AUC for the same duration after oral administration, of between about 1.5 and about 3.0 compared with INVEGA® extended release tablets containing the same amount of Paliperidone administered at the same dose.

8. The dosage form of claim 7, wherein the relative bioavailability is between about 1.7 and about 3.0.

9. The dosage form of claim 8, wherein the relative bioavailability is between about 1.9 and about 3.0.

10. The dosage form of claim 1, wherein the dosage form exhibits a relative Cₘₐₓ after oral administration in human subjects of between about 1.6 and about 3.0 compared with INVEGA® extended release tablets containing the same amount of Paliperidone, administered at the same dose in the human subjects.

11. The dosage form of claim 1 exhibiting an in vitro dissolution profile determined using a 50 RPM paddle method in a dissolution medium of 500 ml 0.05 M phosphate buffer, pH 6.8, wherein the dissolution profile is less than about 10% dissolution in 4 hours, between about 10% to about 25% dissolution in 8 hours, between about 40% to about 60% dissolution in 16 hours and not less than about 70% in 24 hours after the start of the dissolution study, respectively.

12. The dosage form of claim 1, comprising a plurality of particulates, wherein each particulate comprises at least the first component and the second component.

13. The dosage form of claim 12, wherein the second component further comprises coated Paliperidone

14. The dosage form of claim 12, wherein at least some of the particulates are covered by a delayed release layer.

15. The dosage form of claim 12, wherein all of the particulates are covered by a delayed release layer.

16. The dosage form of claim 12, wherein the plurality of particulates comprises a first population of particulates and a second population of particulates, wherein said first population differs from said second population in at least one of 1) weight ratio between said first component and said second component; 2) weight ratio between coated and non-coated paliperidone, 3) weight ratio between paliperidone and other components in the particulate; 4) nature and thickness of coating layer; 5) existence and relative weight of a core and/or sub layer; 6) existence of a second delay layer and the weight ratio between the layers.

17. The dosage form of claim 12, wherein the at least one polymer of the delay layer is selected from the group consisting of hydroxypropyl cellulose, hydroxypropyl methyl cellulose, ethyl cellulose and polymethacrylates.

18. The dosage form of claim 12, wherein the delay layer further comprises at least one plasticizer.

19. The dosage form of claim 18, wherein the plasticizer is hydrophilic.

20. The dosage form of claim 19, wherein the hydrophilic plasticizer is selected from triethyl citrate and polyethylene glycol; and the hydrophobic plasticizer is selected from diethyl phthalate, dibutyl phthalate, dibutyl sebacate and acetyl tributyl citrate.

21. The dosage form of claim 12, each particulate further comprises an inert core covered by a layer comprising Paliperidone or a salt thereof.

22. The dosage form of claim 21, wherein the inert core is selected from the group consisting of microcrystalline cellulose spheres, sugar spheres, or glass spheres.

23. The dosage form of claim 21, wherein the layer comprising Paliperidone or a salt thereof covering the core further comprises at least one pharmaceutically acceptable excipient that acts as a binder.

24. The dosage form of claim 12, wherein the particulates differ in the onset time of paliperidone release and in the rate of release after said onset time.

25. A process for preparing the dosage form of claim 12, comprising:
preparing a particulate by coating a core with a layer comprising non-coated Paliperidone or a salt thereof and applying an extended release layer thereon.

26. The process of claim 25, wherein the layer comprising non-coated Paliperidone or a salt thereof is applied by a process comprising coating the core with a non-coated Paliperidone containing layer using a solution or dispersion of Paliperidone or a salt thereof and a binder.

27. The process of claim 25, wherein the coating process is performed using a fluidized bed coater.

28. The process of claim 27, wherein the fluidize bed coater is equipped with a bottom or top spray device.

29. The dosage form of claim 1, comprising a plurality of particulates wherein each of the particulates comprises at least three layers:
a first layer comprising Paliperidone or a salt thereof and a polymer;
a second layer being a delay release layer covering the first layer; and
a third layer comprising Paliperidone and a polymer.

30. The dosage form of claim 29, further comprising an acid comprising layer, wherein the second layer is a pH-sensitive layer.

31. The dosage form of claim 30, wherein the acid comprising layer comprises at least one organic acid.

32. The dosage form of claim 31, wherein the at least one organic acid is selected from ascorbic acid, tartaric acid and fumaric acid.

33. The dosage form of claim 29, wherein the first layer comprises a core substantially enveloped by the second component, and wherein the core is covered by a layer comprising Paliperidone or a salt thereof.

34. The dosage form of claim 33, wherein the layer that covers the core comprises the Paliperidone or the salt thereof and at least one pharmaceutically acceptable excipient that acts as a binder.

35. The dosage form of claim 34, wherein the binder is selected from methyl cellulose, hydroxypropyl cellulose, hydroxypropyl methyl cellulose and povidone, copovidone, starch, Arabic gum, acasia gum and gelatin.

36. The dosage form of claim 33, wherein the core is an inert core.

37. The dosage form of claim 36, wherein the inert core is selected from microcrystalline cellulose spheres, sugar spheres and glass spheres.

38. The dosage form of claim 29, wherein more than half of the Paliperidone in the third layer is released before the release of a fraction of Paliperidone in the first layer.

39. The dosage form of claim 30, wherein the pH sensitive delay layer will not dissolve until the acid comprising layer is released from the dosage form completely, so that the release of the inner Paliperidone layer is controlled.

40. Paliperidone extended release tablet in the form of an inlay tablet comprising:
(a) an inlay core comprising non-coated Paliperidone and at least one polymer capable of delaying the release of Paliperidone from the inlay core and capable of swelling upon hydration; and
(b) an outer layer comprising a pharmaceutical excipient which is substantially water insoluble, wherein the outer layer partially surrounds the inlay core.

41. The extended release tablet of claim 40, wherein the at least one polymer of the inlay core is selected from the group consisting of polyvinylpyrrolidone, poly(ethylene oxide), POLYOX WSR-301, polysaccharides, hydrophilic cellulose derivatives, methyl cellulose, ethyl cellulose, hydroxypropyl methylcellulose, hydroxyethyl cellulose, hydroxypropyl cellulose, hydroxyethyl methylcellulose, carboxy methylcellulose and sodium carboxy methylcellulose.

42. The extended release tablet of claim 40, wherein the at least one polymer of the inlay core is selected from the group consisting of methyl cellulose, ethyl cellulose, hydroxypropyl methylcellulose, hydroxyethyl cellulose, hydroxypropyl cellulose, hydroxyethyl methylcellulose, carboxy methylcellulose and sodium carboxy methylcellulose.

43. The extended release tablet of claim 40, wherein the at least one polymer of the inlay core is selected from the group consisting of POLYOX WSR-301 and hydroxypropyl methylcellulose.

44. The extended release tablet of claim 40, wherein the at least one polymer of the inlay core is selected from the group consisting of POLYOX WSR-301 and METHOCEL K15MP.

45. The extended release tablet of claim 40, wherein the pharmaceutical excipient which is substantially water insoluble of the outer layer is a pharmaceutically acceptable polymer which is substantially water insoluble.

46. The extended release tablet of claim 45, wherein the pharmaceutically acceptable polymer which is substantially water insoluble is selected from the group consisting of cationic copolymers of ethylacrylate and methylacrylate with quarternary ammonium groups, EUDRAGIT RS, EUDRAGIT RL, ethylacrylate methylmethacrylate copolymer with neutral ester groups, cellulose esters, cellulose ethers and cellulose esterethers, ethyl cellulose, ETHYL CELLULOSE T10 PHARM, cellulose acetate, cellulose diacetate, cellulose triacetate and polyester polymers, poly(ε-caprolactone)s, poly(alkylene glycol adipate)s, poly(ethylene glycol adipate), poly(propylene glycol adipate) and poly(butylene glycol adipate), polyvinyl acetate and blends and copolymers thereof.

47. The extended release tablet of claim 46, wherein the pharmaceutically acceptable polymer which is substantially water insoluble is selected from the group consisting of POLYOX WSR-301 and ETHYLCELLULOSE T10 PHARM.

48. The extended release tablet of claim 46, wherein the pharmaceutically acceptable polymer which is substantially water insoluble is ethyl cellulose.

49. The extended release tablet of claim 40, wherein the inlay core is in the form of a tablet or compressed slug.

50. The extended release tablet of claim 40, wherein the inlay core and/or the outer layer independently further comprise at least one other pharmaceutical excipient.

51. The extended release tablet of claim 50, wherein the at least one other pharmaceutical excipient is selected from the group consisting of pharmaceutically acceptable fillers, diluents, pH modifiers, glidants, lubricants, binders, dyes and flavoring agents.

52. The extended release tablet of claim 40, wherein
the inlay core comprises Paliperidone in about 1-3% w/w, a filler in about 2-5% w/w, pH modifier in about 5-15% w/w, release modifying polymer in about 5-20% w/w, lubricant in about 0-1% w/w and glidant in about 0-1% w/w; and
the outer layer comprises a release modifying polymer in about 50-90% w/w, dye in about 0-1% w/w and lubricant in about 0-1% w/w.

53. The extended release tablet of claim 52, wherein
the inlay core comprises Paliperidone in about 1-3% w/w, STARLAC in about 2-5% w/w, magnesium carbonate USP in about 5-15% w/w, POLYOX WSR-301 in about 5-20% w/w, stearic acid NF in about 0-1% w/w and silicone dioxide NF in about 0-1% w/w; and
the outer layer comprises ETHYLCELLULOSE T10 PHARM in about 50-90% w/w, Ferric Oxide Yellow NF in about 0-1% w/w and stearic acid NF in about 0-1% w/w.

54. A process of making the extended release tablet of claim 40, comprising
(1) mixing Paliperidone and at least one polymer capable of delaying the release of Paliperidone and capable of swelling upon hydration; and
(2) partially covering the mixture of step (1) with an outer layer comprising a pharmaceutical excipient which is substantially water insoluble to obtain the extended release tablet in the form of an inlay tablet.

55. The process of claim 54, wherein the mixture of step (1) is compressed into a slug or tablet before step (2).

56. The process of claim 54, wherein the mixture of step (1) is compressed into a slug or tablet before step (2); the slug or tablet is mixed with the at least one polymer capable of delaying the release of Paliperidone and capable of swelling upon hydration to form a mixture; and the mixture is compressed into a tablet before step (2).

57. The process of claim 54, wherein a filler, pH modifier, glidant and/or lubricant is added in step (1).

58. The process of claim 55, wherein the slug or tablet formed by compression is milled before being mixed with the at least one polymer capable of delaying the release of Paliperidone and capable of swelling upon hydration to form the mixture to be compressed into a tablet before step (2).

59. The process of claim 54, wherein the product of step (2) is compressed to obtain the inlay tablet.
